(19) United States
(12) Patent Application Publication
(10) Pub. No.: US 2016/0058490 A1
(43) Pub. Date: Mar. 3, 2016

(54) INTEGRATED SUTURE AND CAUTERIZATION

(71) Applicants: Robert Hotto, Carlsbad, CA (US); Paul H. Chen, Carlsbad, CA (US)

(72) Inventors: Robert Hotto, Carlsbad, CA (US); Paul H. Chen, Carlsbad, CA (US)

(21) Appl. No.: 14/939,122

(22) Filed: Nov. 12, 2015

Related U.S. Application Data
(62) Division of application No. 13/215,407, filed on Aug. 23, 2011.

(51) Int. Cl. A61B 18/08; A61B 18/14; A61B 18/10

(52) U.S. Cl. CPC A61B 18/08; A61B 18/10; A61B 18/1442; A61B 2018/00595

(57) ABSTRACT

A system for suturing and cauterization is provided. A needle assembly and/or suture line emanates heat to cauterize tissue during wound closure. Energy sources for the heat include thermal elements of a variety of configurations energized from electrical, RF or chemical sources disposed internally or external to the needle assembly. Conductive suture lines are provided and some embodiments include a surgical robot. Wound closure is improved and closing time decreased while the potential for bleeding induced by needle tract incisions and suture tension is minimized.

### TECHNICAL FIELD

This invention relates to suturing and cauterizing devices and systems for employment in the fields of surgery and medicine.

### BACKGROUND

Bleeding is concomitant to many surgical procedures, including, for example, neurological, skin, cardiothoracic, vascular, and abdominal surgery. Surgical bodily repair typically requires bodily tissue incision before targeted areas are reached. Bleeding inevitably ensues. Bleeding adds a risk quotient to surgery and presents in a variety of modes with variable predictability. Consequently, bleeding control is part of the standard repertoire of the surgeon.

A variety of tactical procedures and instruments have, therefore, been devised to reduce unwanted bleeding during surgical procedures. Those prior procedures and instruments have, however, typically contemplated bleeding control as a discrete or separate step in surgical procedure. Separate cauterization of any bleeding in the suture tract takes additional time and risks cutting the suture.

In other instances, specialized tools such as, for example, cauterizing staplers have been employed to minimize bleeding during closing. Surgical staplers are, however, limited. They are more cumbersome than sutures and cannot be used in many situations such as, for example, on small structures and in confined areas. In addition, surgical staples are less secure than sutures and do not provide a continuous sealed tract as can sutures. Further, staplers can leave a more prominent scar than closure with suture.

Consequently, what is needed is a system for wound closure and cauterization that can improve surgical technique and efficiency yet can be employed in a variety of fields and at various scale with disposable tools. Consequently, the present invention provides instruments and procedures to minimize bleeding while concurrently suturing.

### BRIEF DESCRIPTION OF DRAWINGS

FIG. 1 depicts an embodiment of the present invention that includes an energy source and heating element disposed within a suturing needle assembly.

FIG. 1A is an enlarged depiction of the area of FIG. 1 within the dotted circle A and depicts an enlarged view of a portion of the needle assembly of FIG. 1.

FIG. 2 depicts a system having an energy source configured to provide energy to a needle and suture line combination to selectively induce cauterization in surgical wound areas coincident with or soon after closure.

FIG. 3 depicts use of the system depicted in FIG. 2 to apply energy to suture line that has been placed across a just closed wound.

FIG. 4 depicts an embodiment that provides energy to a surgical needle assembly when at least two chemicals are combined.

FIGS. 5A, 5B, and 5C are various depictions of an alternative embodiment of the present invention in which a heat-generating compound is integrated in or on the suturing line.

FIG. 6 depicts a suture line comprised from a conventional suture line combined with a conductive line and therefore adapted for use with embodiments of the present invention that apply cauterization energy through or to a suture line.

FIG. 7 depicts an embodiment of the present invention including a surgical robot.

### DETAILED DESCRIPTION OF PREFERRED EMBODIMENTS OF THE PRESENT INVENTION

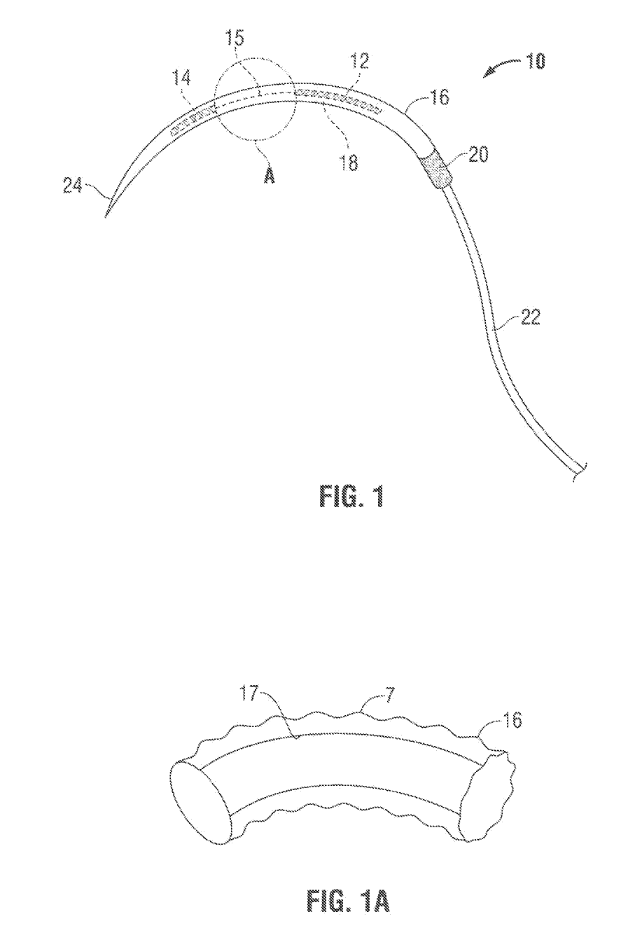

FIG. 1 depicts an embodiment of the present invention. To serve the clarity of the exposition, various features depicted in the Figs. of this disclosure are magnified or are presented in relative scale that differs from real world physical embodiments. Depicted system 10 includes an energy source 12 and a thermal element 14 disposed within a needle assembly 16 thus configured for tissue cauterization and suture. Energy source 12 and thermal element 14 are depicted as connected by conductor pair 15. In some configurations energy source 12 and thermal element 14 may be disposed in contact and conductor pair 15 will be absent. Where there is a separate connective between thermal element 14 and energy source 12, the connection employed between energy source 12 and thermal element 14 may be implemented in a variety of ways and structures such as, for example, a separate conductive wireline as shown as conductor 15 or, alternatively, for example, with a conductive structure along the inner wall 17 of needle assembly 16 as shown in FIG. 1A. In some embodiments, it may be preferable to pass the energy from energy source 12 to thermal element 14 through the body 18 of needle assembly 16. A handling portion 20 of needle assembly 16 may be used to provide a linkage assembly for affixation of suture thread 22 while providing an adjunct handling member for needle assembly 16.

Various modes may be implemented to enable energy source 12. In the embodiment depicted in FIG. 1, energy source 12 is preferably an electrical energy source such as a battery. Surgeon control of thermal emanation from needle assembly 16 can be enabled with a micro-switch or touch activation or thumb control of a SPST switch. In other alternatives, needle assembly 16 may be activated by air exposure when, for example, an air-activated battery, such as a zinc air battery, is employed as energy source 12. Alternatively, energy source 12 may be implemented with a temporary storage device such as a rechargeable battery or slow discharging capacitive element chargeable between uses by, for example, charging power source 12 by placement of needle assembly 16 in an RF cradle.

Thermal element 14 of the embodiment depicted in FIG. 1 preferably produces relatively high heat intensity with minimal energy. Thermal element 14 may be implemented in any of a variety of designs such as, for example, coil or linear structures and may be comprised of heat radiating ceramics or metallic structures with sufficient resistivity to emanate an appropriate level of thermal energy when electrical current is applied. As those of skill will appreciate after understanding this disclosure, the scale employed for various elements of the present invention may be varied across a variety of parameters to suit the intended application both in relevant dimensions such as gauge and material composition.

With continuing reference to FIG. 1, needle assembly 16 includes a piercing portion 24 for tissue penetration. The heat that emanates from thermal element 14 may be preferentially conveyed to piercing component 24 which, as those of skill will recognize, can improve tissue penetration. Alternatively, heat that emanates from thermal element 14 can be preferentially directed further down body 18 to cauterize tissue being closed by suturing with needle assembly 16. Spacing or insulative portion 7 as shown in FIG. 1A may be included in needle assembly 16 to increase thermal isolation of piercing portion 24 and body 18 of needle assembly 16 in embodiments that preferentially project higher levels of thermal energy to either piercing portion 24 or body 18.

As those of skill will understand after appreciation of the present disclosure, several of the described elements may be of one piece or separately fabricated and assembled. For example, as to needle assembly 16, the term “assembly” infers functional features which may be implemented all in one piece or combinations of pieces. Various combinations of elements may be combined in one piece such as, for example, integration of a battery as energy source 12 with thermal element 14. Although disposable configurations are likely to be found most convenient and more readily sterilized, some configurations may provide replaceable power source capability with removal of body 18 of needle assembly 16 from handling member 20 to allow insertion of a new energy source 12 upon exhaustion of the current energy source 12. Further, relative disposition of thermal element 14 and energy source 12 is not limited to any particular relative disposition as, for example, power source 12 may be disposed in the handling portion 20 while the thermal element 14 is disposed in the piercing portion 24 or they may be disposed with various degrees of adjacency.

FIG. 1 depicts energy source 12 as preferably being a source of electrical energy and is an example of embodiments that provide energy to thermal element 14 disposed proximal to an operative portion (e.g., piercing portion 24 in the FIG. 1 depiction) of a surgical needle assembly to precipitate thermal energy release from the piercing portion of the assembly sufficient to cauterize tissue while providing suture based wound closure. However, as stated, the thermal element may be disposed preferentially along the length of needle assembly 16 to preferentially vary the relative time application of cauterization energy relative to tissue penetration. The principle of varying the temporal relationship between closure and cauterization with an embodiment of the present invention can be applied with more variability as disclosed in later embodiments configured to apply energy to conductive suture line after closure. As those of skill will appreciate, cauterization is a matter of degree and when combined with the mechanical closure flexibility allowed with suture (e.g., workable with small field requirements and wide tissue strength and scale range) undesired bleeding and bleeding precipitated by suture stress are ameliorated.

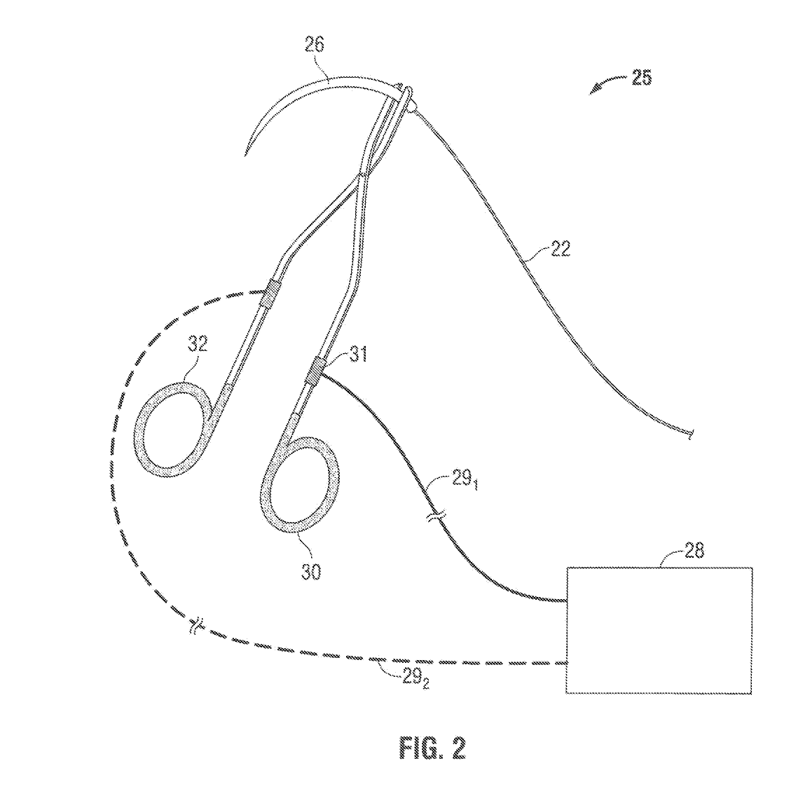

FIG. 2 depicts an alternative embodiment that provides energy to a surgical needle 26 to precipitate controlled cauterization of tissue concomitant with or soon after wound closure. Unlike FIG. 1, in the embodiment of FIG. 2, the energy source is separate from the needle thus providing opportunities to use the principles of the invention with needle structures and suture line of smaller gauge as well as energy sources such as RF that can't be readily generated from within the needle assembly 16. FIGS. 1 and 2 are, however, examples of embodiments of a suturing system configured to release energy, such as thermal or RF energy, for example, from a needle assembly to cauterize surgical wounds while providing mechanical closure through suture.

FIG. 2 depicts an embodiment of the present invention in which the energy source is external to the needle assembly. Depicted system 25 comprises energy source 28 that provides energy along feed line 29₁ to a needle 26 through clamp 30 to cause emanation of energy from desired portions of needle 26 or a conductive suture line 22.

Energy source 28 may be an electrical power supply or a radio frequency (RF) generator. The depiction of FIG. 2 illustrates energy source 28 configured as an RF generator to apply RF to clamp 30 through line 29₁. Conduction line 29₁ is depicted as a single conductor. An energy return path is provided by either an optional return line 29₂ or by use of a ground plate in contact with the patient which is not shown but commonly used in practice.

Clamp 30 is depicted as a needle holder but may be any configuration of clamp, needle holder or forceps or other affixation device to allow manipulation of needle 16. Although the surgeon typically uses gloves, clamp 30 is preferably provided with a nonconductive section 32 on finger loops to suppress RF conduction into the practitioner's hands. For example, the handling portion of clamp 32 may be, for example, plastic.

Line 29₁ is selectively attached to clamp 30 by a selectively attachable collar 31 although such attachment is a matter of design choice with many options available as is recognized by those of skill in the art. Energy source 28 is preferably a generator that produces radio frequency energy of appropriate frequency and Intensity whose energy can be conveyed along conduction path 29₁. Energy source 28 is further preferably operator controlled and a variety of control apparatus are known in the art such as foot or thumb controlled switches to vary the intensity of energy source 28 as deemed appropriate by the practitioner. Thus, FIG. 2 depicts a system having an energy source configured to provide energy to a needle and suture line combination to selectively and controllably induce cauterization in surgical wound areas coincident with or soon after closure.

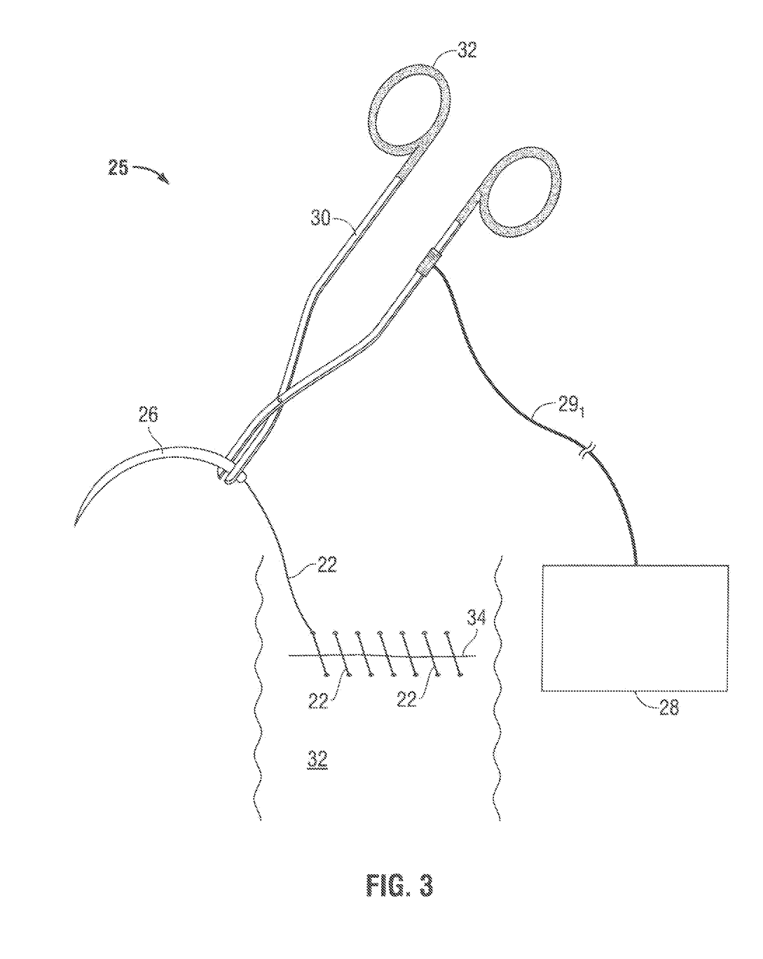

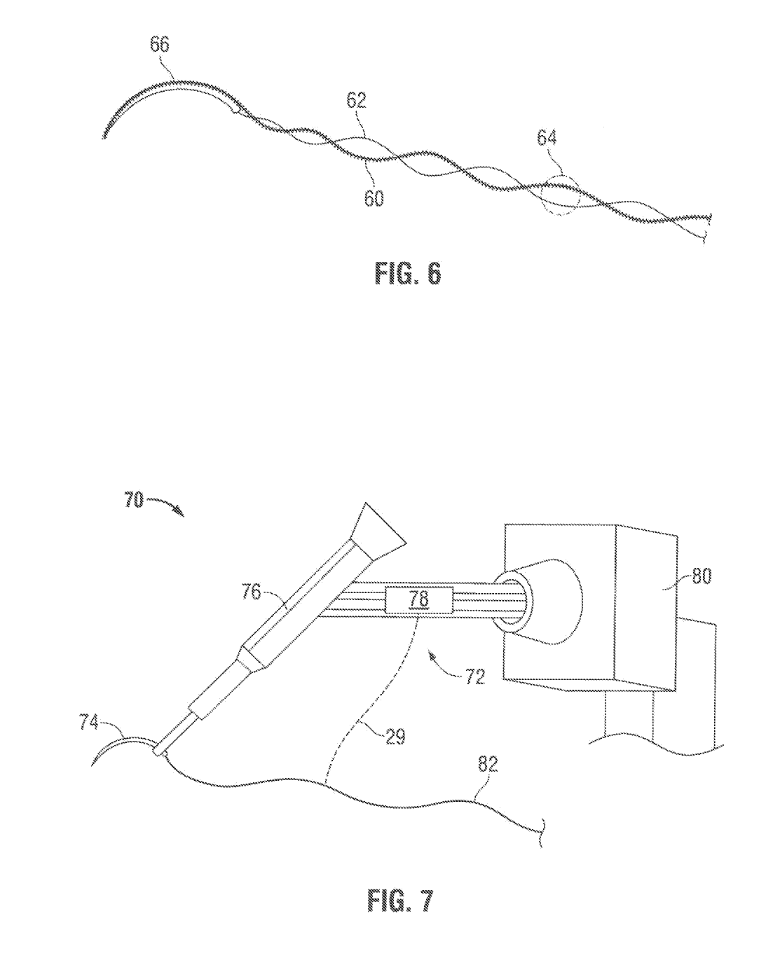

RF structure principles such as, for example, waveguide principles depending upon frequencies employed, known in the art may be employed in implementations of the embodiment of FIG. 2 to direct RF energy where desired. The energy may be directed to the needle assembly or in the suture line itself to cause the emanation of RF energy to cauterize while suturing or, as shown in FIG. 3, after closure. In some instances, conduction path 29₁ can be the suture line 22 itself, if RF conductive material is used for wound closure such as the suture line disclosed and depicted herein and shown by exemplar in FIG. 6.

System 25 is depicted in FIG. 3 configured to apply energy through conduction line 29₁ to clamp 30 and thereby needle 26. Needle 26 is connected to suture line 22 in situ along a just-closed wound 34 of surgical field 32. Suture line 22 is conductive. For example, it may be the suture line shown herein in FIG. 6 and therefore configured to emanate energy from suture line 22 when energized by energy source 28. The system of 25 is therefore configured to cauterize wound 34 after closure. Consequently, because cauterization energy is applied by system 25 through the suture apparatus (e.g., the needle and or suture line itself), no separate cauterization device is needed and therefore disturbance of just closed wound 34 is minimized. As those of skill will recognize, by emanation of RF energy, tissue is cauterized and system 25 is configured to provide such cauterization in conjunction with wound closure through suture.

Alternative embodiments of the present invention employ, amongst other alternative structures, chemical compounds having exothermic characteristics to provide energy to cause heat emanation from a surgical needle to realize coincident suturing and cauterization scalable for large or small fields and a variety of suturing thread types and applications. In other embodiments heat is emanated from the suture line itself by way of embedding the suture line itself with thermally-exothermic substances.

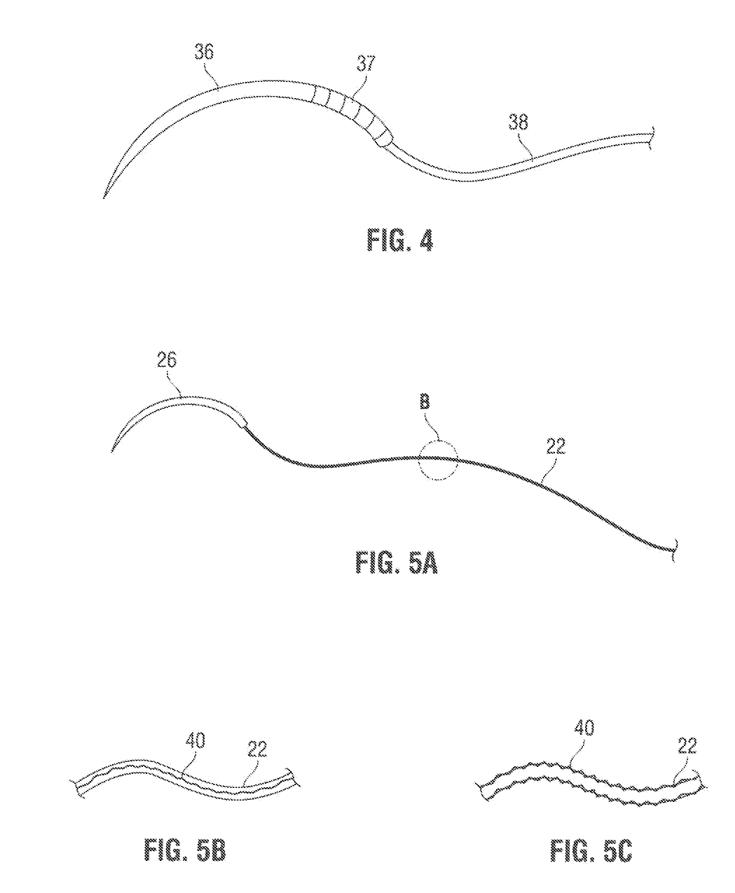

FIG. 4 depicts an embodiment that provides energy to a surgical needle assembly 36. Needle assembly 36 is configured with a chemical mixture of at least two chemicals mixed by breaking a barrier in section 37 of needle assembly 36 with, for example, a clamp. The resulting exothermic reaction directs released thermal energy into the operative portion of needle assembly 36 to cauterize tissue while affixing suture line 38 across the targeted surgical opening. An alternate embodiment employs an exothermic chemical reaction such as comprising a mixture of iron, water, cellulose, vermiculite, activated carbon and salt. Such embodiments are more suitable to field operations where expediency is a high value and typical surgical theater infrastructure is not available.

FIGS. 5A, 5B, and 5C depict an alternative embodiment of the present invention in which a heat-generating compound is integrated in or on the suturing line 22 which is connected to surgical needle 26. For example, FIG. 5A has a focus circle marked B which is enlarged in various embodiments shown in FIGS. 5B and 5C. Cauterization agent 40, such as silver nitrate, or iron water, in or on the suture line 22 can release heat sufficient to induce a degree of cauterization coincident with suture closure. Suture line 22 is shown in FIG. 5B with cauterization agent 40 embedded in line 22 while in FIG. 5C, cauterization agent 40 is present on the surface of line 22. Each of these embodiments are likely to find more useful employment in field applications when well-fitted surgical theaters are not available.

FIG. 6 depicts a suture line comprised from a traditional surgical thread 60 wound with a conductive line 62 to create a suture line 64 affixed to needle 66. Suture line 64 is configured for use in conjunction with, for example, the systems shown in the present disclosure. Traditional thread 60 includes any of the wide range of suture lines available and known in the art including, just as examples, dissolving line or more rugged lines for heavier tissue applications. The conductive line 62 of suture line 64 may be light gauge metallic material or other conductive elements such as conductive plastics which are known in the art.

FIG. 7 depicts an embodiment of the present invention. Depicted system 70 includes surgical robot 72 that applies RF energy to needle 74 to cauterize a surgical wound in coincidence with closure. Robotic arm 76 is highly controlled from base 80 to perform surgery of high precision. Energy supply 78 provides RF energy to needle 74 or, preferentially, it may apply RF energy to suture line 82, if conductive as depicted by optional connective line 29. Energy supply 78 may also be external to the robot. The use of a robot enables precise and very small suturing and cauterization on small structures and in confined areas with precision that is difficult for a human to perform consistently. In addition, the robot can apply RF energy intensities in levels that exceed levels acceptable for a human operator.

What is claimed is:

1. Device comprising:
a surgical needle formed with a distal tip that is pointed to pierce tissue;
a suture line engaged with the surgical needle; and
a heat-generating compound integrated with the suture line.

2. The device of claim 1, wherein the heat-generating compound includes silver nitrate.

3. The device of claim 1, wherein the heat-generating compound includes iron water.

4. The device of claim 1, wherein the heat-generating compound is embedded in the suture line.

5. The device of claim 1, wherein the heat-generating compound is disposed on the surface of the suture line.

6. The device of claim 1, wherein the heat-generating compound includes a conductive line wound around the suture thread and connectable to an energy source to energize the conductive line for tissue cauterization.

7. The device of claim 6, wherein the conductive line is made of light gauge metallic material.

8. The device of claim 6, wherein the conductive line is made of conductive plastic.

9. The device of claim 6, comprising the energy source.
